(19) United States
(12) Patent Application Publication
(10) Pub. No.: US 2003/0124192 A1
(43) Pub. Date: Jul. 3, 2003

(54) Pharmaceutical compositions and their preparation

(76) Inventors: Colin Stanley Fitchett, Hertfordshire (GB); Jodie Hadley, Hertfordshire (GB); Richard Le Page, Cambridge (GB); James Good, Cambridge (GB); Christopher Coward, Cambridge (GB)

Correspondence Address: Heller Ehrman White & McAuliffe, Suite 300, 1666 K Street N W, Washington, DC, 20006, US

(21) Appl. No.: 10/169,407

(22) PCT Filed: Dec. 22, 2000
(86) PCT No.: PCT/GB00/04994

(30) Foreign Application Priority Data
Jan. 5, 2000 (GB) 0000001.8

(51) Int. Cl. A61K 31/70

(52) U.S. Cl. 424/489; 514/23

(57) ABSTRACT

The invention provides the use of a hemicellulose for the manufacture of a composition for use as a vaccine adjuvant. The hemicellulose is preferably an arabinoxylan and more preferably is a crosslinked arabinoxylan such as arabinoxylan ferrulate. Also provided by the invention are vaccine compositions containing a vaccine antigen, or a DNA vaccine, and the hemicellulose, preferably in crosslinked microparticulate form.

### FIELD OF THE INVENTION

This invention relates to compositions, such as pharmaceutical compositions, comprising hemicelluloses, and to the use of hemicelluloses as vaccine adjuvants and pharmaceutical delivery systems.

### BACKGROUND OF THE INVENTION

The development of delivery systems to deliver antigens, such as DNA and proteins, to target molecules to the immune system is of prime importance in optimising vaccine performance. A delivery system is a physical entity which carries molecules to particular compartments in vivo. If the antigen is made particulate rather than soluble, the immune system encounters the antigen as it would during an infection since bacterial and viral antigens are particulate. Microparticles can be trapped and retained in lymph nodes more readily than can soluble antigens promoting effective antigen processing and cytokine release (these are all events that are known to promote the induction of immune responses). It has been demonstrated that microparticles ranging from 1-5 μm in size are taken up by macrophages, a type of cell that is particularly involved in immunity. An effective microparticulate delivery system in general should have a mean particle size of 1 μm or less (Saunders et al., Int. J. Pharm. 68, 265-270, 1991).

Examples of known microparticulate delivery vehicles include poly-lactide-co-glycolide (PLG) polymers which, when used appropriately, can elicit antibodies and cell-mediated immunity, and can be taken up from mucosal surfaces. However, PLG polymers are difficult to prepare quickly and routinely, and require exposure of antigens to organic solvents.

One object of the present invention therefore is to provide a microparticulate delivery vehicle for vaccine antigens that can be prepared more easily than known polymers such as the PLG vehicle referred to above. A further object of the invention is to provide a microparticulate delivery system which can be used to deliver a wide range of pharmaceutical substances in addition to vaccines.

U.S. Pat. No. 5,174,998 (Nisshin Flour Milling Co. Ltd) discloses the use of a hemicellulose extracted from wheat bran or a wheat bran extract as a matrix for controlled release of drug substances. However, there is no suggestion that such hemicelluloses can be used to deliver vaccines and there is no suggestion that the hemicelluloses should be crosslinked and presented in particulate form.

Our earlier International patent applications WO-A-98/22513 (the disclosure in which is incorporated herein by reference) discloses that gelling hemicellulose material, or gels or viscous media prepared therefrom may contain an antibiotic, electrolyte, cell, tissue, cell extract, pigment, dye, radioisotope, label, imaging agent, enzyme, co-factor, hormone, cytokine, vaccine, growth factor, protein (e.g. a therapeutic protein), allergen, hapten or antigen (for e.g. sensitivity testing), antibody, oil, analgesic and/or antiinflammatory agent (e.g. NSAID). However, no reference is made to the formation of microparticulates nor is there any suggestion that the microparticulates may have adjuvant activity.

Hemicelluloses are obtained from plant tissue, especially cell wall material. The term “hemicellulose” is a term of art used to embrace non-cellulosic, non-starch plant polysaccharides. The term therefore embraces inter alia pentosans and pectins.

Some hemicelluloses are suitable as substrates for oxidative gelation (“gelling hemicelluloses”): such hemicelluloses often have substituents with phenolic groups which are cross-linkable with certain oxidizing agents.

Arabinoxylan and pectin constitute two particularly important classes of hemicellulose. Arabinoxylans consist predominantly of the pentoses arabinose and xylose, and are therefore often classified as pentosans. However, in many cases hexoses and hexuronic acid are present as minor constituents, and therefore they may also be referred to descriptively as heteroxylans.

The arabinoxylan molecule consists of a linear backbone of (1-4)-β-xylopyranosyl units, to which substituents are attached through the O-2 and O-3 atoms of the xlosyl residues. The major substituents are single α-L-arabinofuranosyl residues. Single α-D-glucuronopyranosyl residues and their 4-O-methyl ethers are also common substituents.

Arabinoxylan preparations are usually heterogenous with respect to the ratio of xylose to arabinose (i.e. the degree of substitution) and in the pattern of substitution of the arabinosyl units along the (1-4)-β-xylan backbone.

Phenolic acid (including ferulic acid) and acetyl substituents occur at intervals along the arabinoxylan chains. These substituents to some extent determine the solubility of the arabinoxylan. Arabinoxylan preparations bearing phenolic (e.g. ferulic acid substituents) are referred to herein as “AXF”, while those bearing acetyl substituents are designated “AXA”. Similarly, preparation bearing both phenolic (e.g. ferulic acid) and acetyl substituents are hereinafter abbreviated to the designation “AXFA”. Arabinoxylan preparations having few phenolic (e.g. ferulic acid) substituents are designated “AX”: when the degree of substitution falls below that required for oxidative gelation, the arabinoxylan is designated a “non-gelling arabinoxylan” (a term which therefore embraces AX and AXA).

Pectins constitute another important class of hemicelluloses. As used herein and unless otherwise indicated, the term “pectin” is used sensu lato to define hemicellulose polymers rich in D-galacturonic acid. Many (but not all) are cell wall components. The term “pectin” is also used herein sensu stricto to define the so-called “true pectins”, which are characterised by the presence of an O-(α-D-galacturonopyranosyl)(1-2)-L-rhamnopyranosyl linkage within the molecule.

The pectins may be subcategorized on the basis of their structural complexity. At one extreme are “simple pectins”, which are galacturonans. At the other extreme are “complex pectins” exemplified by rhamnogalacturonan II, which contains at least 10 different monosaccharide components in the main chain or as a components of branches. Pectins of intermediate complexity (herein referred to as “mesocomplex pectins” contain alternate rhamnose and galacturonic acid units, while others have branches of glucuronic acid linked to galacturonic acid.

Complex and mesocomplex pectins are made up of “smooth” regions (based on linear homogalacturonan) and “hairy” regions corresponding to the rhamnogalacturonan backbone with side-branches of varying length.

Certain pectins (for example, pectins obtainable from representatives of the plant family Chenopodiaceae, which include beets (e.g. sugar beet), spinach and mangelwurzels) are substituted to some extent with substituents derived from carboxylic acids (usually substituted cinnamic acids) containing phenolic groups. Such pectins may be oxidatively cross-linked to produce viscous solutions or gels via their phenolic substituents. This can be achieved by powerful oxidants (e.g. persulfate-see J. -F. Thibault et alia, in The Chemistry and Technology of Pectin, Academic Press 1991, Chapter 7, pages 119-133) or a combination of peroxidase and hydrogen peroxide (see Thibault et alia, ibidem). FR 2 545 101 A1 also describes the gelling of beet pectins using an oxidant (e.g. hydrogen peroxide) and an enzyme (peroxidase). Such pectins are referred to herein as “gelling pectins”.

Sugar beet pectin is especially rich in arabinan. Arabinan contains β-1, 5-linked arabinose in the backbone with α-(1→3) or α-(1→2)-linked arabinose residues, whereas arabinogalactan contains β-1, 4-linked galactose in the backbone, with α-(1→3) or α-(1→2) linked arabinose residues. Ferulyl substituents are linked to the arabinose and/or the galactose in the arabinan and arabinogalactan side-branches of the rhamnogalacturonan part. The “ferulic acid” content varies according to the extraction method, but is often about 0.6%.

Beet pectins obtained by processes which partially remove arabinose residues may exhibit improved gelling properties. Thus, procedures involving mild acid treatment and/or treatment with an α-arabinofuranosidase will improve the gelling properties of the pectin (see F. Guillon and J. -F. Thibault, ibidem). Such pectins are hereinafter referred to as “treated pectins”. Hemicelluloses are complex mixtures of noncellulosic cell wall polysaccharides, including pentosans such as arabinoxylans. Convenient sources of hemicelluloses include cereals (such as maize, barley, wheat, oats, rice), pulses (e.g. soya), legumes and fruit.

There are many known methods for fractionating plant material (such as testaceous or cell wall material) to produce hemicellulose and cellulose fractions. Such methods usually involve alkali or water extraction to yield insoluble cellulose and soluble hemicellulose fractions, followed by separation. The soluble extract is then often neutralized (or acidified) to precipitate hemicelluloses. Organic solvents are also commonly used instead of (or in addition to) acidification to precipitate further hemicellulose fractions.

Aqueous extracts of many hemicellulose fractions are known to form gels (or viscous media) when treated with oxidizing agents. The biochemical basis of the gelling process is not yet fully understood. However, gel formation is thought to arise (at least in part) from cross linking within and/or between macromolecular components of the hemicellulose mediated by ferulic acid residues (for example, involving diferulate generated by oxidative coupling of the aromatic nucleus of ferulic acid). These ferulic acid residues occur on arabinoxylans present in the hemicellulose. Extensive hydrolysis (by e.g. harsh alkaline treatments) is known to strip the ferulic acid residues from the bulk pentosans, and so hemicelluloses for use as starting materials in the production of gels or viscous solutions are usually extracted by water (particularly hot water) or mild alkali extraction.

As used herein (and as is usual in the art), the terms “ferulic acid” and “ferulate” are used sensu lato to encompass ferulyl (often denoted feruloyl) groups (i.e. 4-hydroxy-3-methoxy-cinnamyl groups) and derivatives (particularly oxidized derivatives) thereof.

Only a few oxidizing agents are known to have the ability to induce gelation, and these include hydrogen peroxide (usually in conjunction with a peroxidase), ammonium persulphate and formamidine disulphide. WO 96/03440 describes the use of an oxidase (preferably a laccase) for promoting oxidative gelation of inter alia arabinoxylans. WO 93/10158 describes oxidative gelation of hemicellulosic material using an oxidizing system comprising a peroxide (such as hydrogen peroxide) and an oxygenase (such as a peroxidase). Our earlier application WO98/22513 discloses that hemicelluloses can be gelled using an oxidase (e.g. glucose oxidase) and optionally a peroxidase (e.g. horse radish peroxidase).

### SUMMARY OF THE INVENTION

The present invention relates to new uses for hemicelluloses, particularly crosslinked gelled hemicelluloses, and to new compositions derived from hemicelluloses.

According to one aspect of the present invention, there is provided the use of a hemicellulose for the manufacture of a composition for use as a vaccine adjuvant.

In another aspect, the invention provides the use of a hemicellulose for the manufacture of a composition for enhancing the immune response of an antigen co-administered therewith.

The hemicellulose can be as described above but typically is a gelling or gelled hemicellulose. More usually, the hemicellulose is crosslinked, for example oxidatively crosslinked.

The hemicellulose is preferably an arabinoxylan, more preferably an arabinoxylan substituted by crosslinkable groups such as ferulate groups as hereinbefore defined.

The inventors have found that uncrosslinked hemicellulose, particularly uncrosslinked arabinoxylan, has adjuvant activity when co-administered with a vaccine antigen, but that crosslinking the AXF significantly enhances the adjuvant activity.

The crosslinked cellulose is most preferably in the form of microparticulates since it has been found that presentation of the hemicellulose as microparticulates enhances the adjuvant properties of the hemicellulose.

Accordingly, in another aspect, the invention provides a composition comprising a crosslinked hemicellulose as hereinbefore defined, the crosslinked hemicellulose being in the form of microparticles having a mean particle size of less than 50 μm (preferably less than 30 μm, more preferably less than 20 μm, for example less than 10 μm, e.g. less than 5 μm).

In a further aspect, the invention provides a pharmaceutical composition comprising a crosslinked hemicellulose as hereinbefore defined, the crosslinked hemicellulose being in the form of microparticles having a mean particle size of less than 50 μm (preferably less than 30 μm, more preferably less than 20 μm, for example less than 10 μm, e.g. less than 5 μm), the microparticles containing or having associated therewith a pharmaceutically useful substance such as a vaccine or a drug.

In order to facilitate delivery of vaccine substances such as vaccine antigens and DNA, it is most preferred that the microparticles have a mean particle size in the range 0.5 μm to 2 μm, for example approximately 1 μm.

In one preferred embodiment, there is provided a pharmaceutical composition as hereinbefore defined which is a vaccine composition, the active substance being a vaccine antigen or a nucleic acid molecule (e.g. DNA encoding a vaccine antigen) capable on expression of eliciting an immune response.

The vaccine antigens can be, for example, antigens derived from viruses, bacteria and protozoa. Thus, for example, the antigens can be derived from viruses such as influenza, human immuno-deficiency virus (HIV), cytomegalovirus (CMV), vesicular stomatitis virus, respiratory syncytial virus (RSV), herpes viruses such as herpes simplex and herpes zoster, human papilloma virus (HPV), Epstein-Barr virus (EBV), rhinoviruses, hepatitis viruses such as hepatitis A, B or C, adenoviruses, paramyxoviruses and myxoviruses, picornaviruses. Examples of bacterial antigens include antigens derived from Bordetella pertussis, Helicobacter pylori, Streptococcus spp, Salmonella spp, Staphylococcus spp, Mycobacterium spp, Vibrio cholerae, Meningococcus spp, Escherischia spp e.g. E. coli, particular examples being antigens from pertussis, diphtheria, tetanus, cholera, and salmonella. Examples of protozoal antigens include malarial circumsporozoate antigens.

The vaccine antigens can be in the form of whole cells or whole viruses, either killed or attenuated (for example attenuated Salmonella spp), or antigenic fragments or purified or partially purified antigens (for example purified peptides, or lipopolysaccharides). Alternatively, the antigens can be wholly or partially synthetic in nature or can be formed by recombinant techniques. Thus, for example, the antigens can be produced by genetic engineering methods as fusion proteins with a carrier protein or one or more other antigens, for example antigens from other microoorganisms.

Instead of, or in addition to, including antigens, the vaccine can be a DNA vaccine in which the vaccine substance is a DNA molecule coding for an antigenic substance. Examples of DNA vaccines include vaccines containing the DNA coding for hepatitis B surface antigen, Herpes simplex glycoprotein, HIV envelope and regulatory proteins, influenza haemagglutinins, malaria circumsporozoite protein, carcinoembryonic antigen (CEA), prostate specific membrane antigen, and T-cell receptor.

When the vaccine is a DNA vaccine, it is presently preferred that the DNA is not encapsulated within the AXF. Instead, it can for example be formulated as a mixture with the AXF (e.g. AXF in the form of particles such as microspheres of crosslinked AXF) and co-administered therewith. Alternatively, it can be formulated and administered separately from the AXF.

In addition to being useful as vaccine delivery vehicles and adjuvants, microparticles formed from hemicellulose according to the invention can also be used as vehicles for the delivery of non-vaccine substances. Such substances can be for example, biologically active substances for use in therapy (or in the prophyllaxis of disease), or may be diagnostic substances such as labels (e.g. dyes, radioisotopes, or fluorescent labels), or electrolytes, cells, cell extracts, pigments, imaging agents, or enzymes.

Accordingly, in another embodiment of the invention, there is provided a pharmaceutical composition as hereinbefore defined in which the pharmaceutically useful substance is other than a vaccine substance, e.g. is a substance for use in therapy.

Examples of such substances are those selected from anti-inflammatory substances, anticancer agents, antiproliferative agents such as agents for treating psoriasis, vasodilators, adrenergic agents such as α-agonists and β-antagonists, cholinergic agents, antiglaucoma agents, analgesics, antimuscarinics, anti-pyretics, hypnotics, sedatives, migraine treatments, decongestants, urocosurics, diuretics, anti-emetics, anti-arrythmics, anticoagulants, anti-infectives such as antibacterials, antifungals, antivirals and antiprotozoals, antithrombotics, anti-anginal agents, anti-asthmatics, antihistamines, osteoporosis treatments, immunomodulators such as immune stimulators and immune suppressants, vasoconstrictors, agents for treating Parkinsonism and Alzheimer's disease, vitamins, antipyretics, neuromuscular drugs, drugs for treating gastrointestinal disorders such as Crohn's disease, irritable bowel syndrome, and ulcerative colitis, anticonvulsants, antihypertensives, anti-ulcer drugs, anabolic substances, steroidal substances such as corticosteroids, contraceptives, sex hormones, peptide hormones, therapeutic peptides such as inteferons, immunoglobulins, calcitonins, insulin, vasopressins, erythropoietin, plasminogen activators, growth factors, cytokines, allergens and enzymes.

The crosslinked gel microparticles of the invention can be prepared by comminuting a gel either in the hydrated or solution state or following drying. The term “comminuting” as used herein includes treatments such as grinding or milling (e.g. ball milling) but also includes techniques such as dispersion of the gel in a liquid phase, for example by disruptive methods such as vortexing or sonication.

Accordingly, in another aspect, the invention provides a method of preparing crosslinked hemicellulose microparticles (e.g. microparticles suitable for use in a pharmaceutical composition), which method comprises subjecting an uncrosslinked solution of a hemicellulose to gellation and comminuting the gel to give microparticles having a mean particle size of less than 50 μm.

In one embodiment, the gel is dried prior to comminuting, for example by means of freeze drying. The dried gel can then be comminuted using a suitable milling technique, preferably a low shear technique such as ball milling. The advantage of using a low shear technique such as ball milling is that it reduces the likelihood of damage to shear-sensitive molecules such as DNA and RNA.

As an alternative to drying and then milling, the gel can be comminuted whilst in a hydrated state. For example, the gel can be formed or introduced into an aqueous solution, for example a buffered solution, and then dispersed to form microparticulates using sonication or vortexing.

A pharmaceutically useful substance can be mixed with the hemicellulose prior to gelling and comminuting. In this manner, the pharmaceutically active substance can be encapsulated by the hemicellulose during the gellation.

Alternatively, the gel can be dried, either prior to or after comminuting, and a solution of the pharmaceutically useful substance subsequently brought into contact with the dried gel. Thus, for example, the comminuted microparticles of gel can be soaked in a solution of the pharmaceutically active substance such that they are absorbed into the microparticles.

By incorporating the pharmaceutically useful substance into the crosslinked gel particles, the release characteristics of the substance can be modified. Thus, for example, depending on the density of crosslinking, and the molecular size of the pharmaceutically active substance, the crosslinked gel particles of the invention can function as release retarding agents. As such, they can be used in controlled release formulations, for example where a sustained release of an active substance is required over an extended period.

The microparticles of the invention can be used for the delivery of vaccines and hence may contain, or be associated with, vaccine substances such as DNA or vaccine antigens. As such, the microparticles of the invention can act as adjuvants by enhancing the immune response of a host (such as a mammalian host) to the vaccine.

Accordingly, in a still further aspect, the invention provides a method of enhancing the immune response of a host to an antigen, which method comprises administering to the host, in addition to the antigen, a hemicellulose adjuvant as hereinbefore defined.

In a further aspect, the invention provides a method of stimulating an immune response to a vaccine antigen in a host (e.g. a mammalian host), which method comprises administering to the host an effective amount (e.g. an effective immune stimulating amount) of the vaccine antigen and an effective adjuvant amount of a hemicellulose adjuvant as hereinbefore defined.

An advantage of the hemicelluloses and in particular the crosslinked hemicellulose particles of the invention is that although they act as adjuvants, they remain inert at the site of injection of a vaccine (e.g. subcutaneous injection) and are not pro-inflammatory.

The hemicellulose for use in the invention may be any hemicellulose meeting the definition set out earlier. In particular, the hemicellulose may be an arabinoxylan, heteroxylan or pectin. In addition, the hemicellulose for use in the processes of the invention may be a synthetic hemicellulose (i.e. a structural analogue of a naturally-occurring hemicellulose synthesised in vitro by any chemical/enzymic synthesis or modification).

Preferred hemicelluloses are those that are suitable as substrates for oxidative gelation (“gelling hemicelluloses”): such hemicelluloses often have substituents with phenolic groups which are cross-linkable with certain oxidizing agents. Non-gelling hemicelluloses may be first derivatized with phenolic (e.g. ferulic) acid groups prior to use in the invention.

Arabinoxylans, heteroxylans and pectins may also be used. Of the arabinoxylans, particularly preferred are AXFA, AXF, AXA and AX, with AXF being particularly preferred.

The hemicellulosic material may be obtained by any of the standard techniques known in the art for obtaining hemicelluloses suitable as starting materials for oxidative gelation. Preferably, the hemicelluloses are obtained by any of the processes described in WO 93/10158.

The hemicellulosic material may be derived from any of a wide range of different sting materials. Suitable starting materials containing hemicellulose for use in the invention typically include plant material of various kinds and any part or component thereof.

Plant materials useful as a starting material in the invention include the leaves and stalks of woody and nonwoody plants (particularly monocotyledonous plants), and grassy species of the family Gramineae. Particularly preferred are gramineous agricultural residues, i.e. the portions of grain-bearing grassy plants which remain after harvesting the seed. Such residues include straws (e.g. wheat, oat, rice, barley, rye, buckwheat and flax straws), corn stalks, corn cobs and corn husks.

Other suitable starting materials include grasses, such as prairie grasses, gamagrass and foxtail. Other suitable sources include dicotyledonous plants such as woody dicots (e.g. trees and shrubs) as well as leguminous plants.

Another preferred source are fruits, roots and tubers (used herein in the botanical sense). The term “fruit” includes the ripened plant ovary (or group thereof) containing the seeds, together with any adjacent parts that may be fused with it at maturity. The term “fruit” also embraces simple dry fruits (follicles, legumes, capsules, achenes, grains, samaras and nuts (including chestnuts, water chestnuts, horsechestnuts etc.)), simple fleshy fruits (berries, drupes, false berries and pomes), aggregate fruits and multiple fruits. The term “fruit” is also intended to embrace any residual or modified leaf and flower parts which contain or are attached to the fruit (such as a bract). Encompassed within this meaning of fruit are cereal grains and other seeds. Also contemplated for use as starting materials are fruit components, including bran, seed hulls and culms, including malt culms. “Bran” is a component of cereals and is defined as a fraction obtained during the processing of cereal grain seeds and comprises the lignocellulosic seed coat as separate from the flour or meal. Other suitable component parts suitable as starting materials include flours and meals (particularly cereal flours and meals, and including nonwoody seed hulls, such as the bracts of oats and rice).

The term “root” is intended to define the usually underground portion of a plant body that functions as an organ of absorption, aeration and/or food storage or as a means of anchorage or support. It differs from the stem in lacking nodes, buds and leaves. The term “tuber” is defined as a much enlarged portion of subterranian stem (stolon) provided with buds on the sides and tips.

Preferred lignocellulosic staring materials include waste stream components from commercial processing of crop materials such as various beets and pulps thereof (including sugar beet pulp), citrus fruit pulp, wood pulp, fruit rinds, nonwoody seed hulls and cereal bran. Suitable cereal sources include maize, barley, wheat, oats, rice, other sources include pulses (e.g. soya), legumes and fruit.

Other suitable starting materials include pollen, bark, wood shavings, aquatic plants, marine plants (including algae), exudates, cultured tissue, synthetic gums, pectins and mucilages.

Particularly preferred as a starting material is testaceous plant material, for example waste testaceous plant material (preferably containing at least about 20% of arabinoxylan and/or glucoronoarabinoxylan).

The starting material may be treated directly in its field-harvested state or (more usually) subject to some form of pre-processing. Typical pre-processing steps include chopping, grinding, cleaning, washing, screening, sieving etc.

Preferably, the starting material is in a substantially ground form having a particle size of not more than about 100 microns. It may be air classified or sieved (for example to reduce the level of starch). Alternatively, or in addition, the starting material may be treated with enzymes to remove starch (e.g. alpha- and/or beta-amylase). The staring material may also be pre-digested with a carbohydrase enzyme to remove β-glucan.

Suitable washing treatments include washing with hot water or acid (e.g. at a pH of 3-6, e.g. about 5). This at least partially separates protein. Other pre-treatments include protease treatment.

The hemicellulosic material may, for example, be obtained from cereal husk or bran, or legumes, e.g. from maize, wheat, barley, rice, oats or malt, though any source of hemicellulose may be used in the invention so long as it is subject to at least some degree of oxidative gelation. Maize is presently preferred.

Preferably, the hemicellulosic material comprises a pentosan, e.g. a water soluble or alkali soluble pentosan fraction. Particularly preferred are materials wherein the pentosan comprises arabinoxylan, for example arabinoxylan ferulate. In one preferred embodiment, the hemicellulose of the invention consists (or consists essentially) of arabinoxylan ferulate.

Regardless of the source of the hemicellulose, it will generally be necessary to purify the hemicellulose to a level of purity at which it is suitable for use in a pharmaceutical context, i.e. contains no (or substantially no) substances or entities which may have a detrimental effect on the person or other patient to whom they are administered. Thus, for example, the hemicellulose will typically be purified to remove and/or deactivate pyrogens, or allergenic substances, or microbes and their toxins and metabolites, or substances that may interfere with the functioning of any active component carried by or formulated with the hemicellulose. Such purification can be carried out for example, by methods known per se for the purification of hemicelluloses.

The hemicellulosic materials can be gelled by oxidative gellation. For example, gellation can be effected by means of a suitable oxidising agent such as a peroxide (e.g. hydrogen peroxide), optionally in the presence of an oxidase (such as a peroxidase), e.g. as described in WO 93/10158. Alternatively, a process for effecting oxidative gelation of a hemicellulosic material can comprise the step of promoting the generation of hydrogen peroxide in situ by redox enzymes.

In one preferred embodiment, the hemicellulose is gelled by means of an oxidativel crosslinking reaction effected by an oxidase (e.g. glucose oxidase) and optionally a peroxidase (e.g. horse radish peroxidase).

The hemicellulose can be supplemented with an oxidase and optionally an oxidase substrate and/or a peroxidase. The generation of hydrogen peroxide is then preferably promoted by:

(a) providing oxygen to the material (e.g. by generating oxygen in situ); and/or

(b) providing water to the material; and/or

(c) providing oxidase substrate to the material (e.g. by generating substrate in situ);

and/or

(d) activating one or more of the redox enzymes (e.g. chemically or physically), wherein the provision of oxygen or substrate may be by controlled release or generation in situ, for example triggered generation or release by heat, irradiation or chemical treatment.

Pharmaceutical Formulations

The compositions (e.g. cross-linked hemicellulose microparticle) compositions of the invention can be formulated in a variety of ways, depending inter alia on their intended uses. For example, they can be formulated for administration orally, sublingually, parenterally, transdermally, intramuscularly, subcutaneously, rectally, vaginally, intranasally, intrabronchially, via inhalation or via buccal administration. As such, the compositions of the invention can be presented as solutions, syrups, tablets, capsules, lozenges, inserts, patches, powders, pills, solutions for injection or drops, or aerosols such as dry powder aerosols or liquid aerosols, by way of example. Such formulations can be prepared in accordance with methods well known per se.

Vaccine compositions can be formulated for inter alia administration by subcutaneous, intradermal, intramuscular, intra-bronchial, intra-nasal, intravenous, intra-peritoneal or oral delivery. Thus, the compositions can be administered in the form of solutions or fine suspensions (in the case of the hemicellulose micro-particles) for injection or administration as sprays or drops, for example aerosols for inhalation or nasal drops. Alternatively, the compositions can be administered as dry powders, e.g. for inhalation.

Liquid compositions will generally be prepared in sterile water, for example water for injection, and will preferably be substantially free of pyrogens and other unwanted and/or potentially harmful substances such as endotoxins. Where the compositions are for administration by injection or infusion, they will typically be presented in the form of filtered sterile solutions, preferably in physiological saline buffered to approximately pH7. Alternatively, they can be presented as sterile powders for making up into injectable or infusible solutions.

The compositions may contain other vaccine excipients and auxiliary substances, for example one or more excipients of the type usually included in such compositions such as preservatives, viscosity adjusting agents, tonicity adjusting agents, buffering agents and the like. The antigen or mixture of antigens typically is selected such that it is non-toxic to a recipient thereof at concentrations employed to elicit an immune response.

Non-vaccine compositions can be presented in a wide range of different formulation types and can, for example take the form of suspensions, powders, and solid or semi-solid unit dosage forms such as tablets, granules, lozenges or capsules.

A solid or semi-solid dosage form according to the present invention can contain, for example, from 10 mg to 1000 mg of a biologically active substance (preferably a therapeutically useful substance) as hereinbefore defined, more typically 50 mg to 500 mg, e.g. 100 mg to 400 mg, and in particular 150 mg to 350 mg, particular unit dosages being approximately 200 mg and 300 mg.

A tablet composition may contain, in addition to the hemicellulose microspheres of the invention, one or more pharmaceutically acceptable solid diluents or compression aids, examples of which include sugars such as sucrose and lactose, and sugar alcohols such as xylitol, sorbitol and mannitol; lactose and sorbitol being particular examples.

The tablets may also contain one or more excipients selected from granulating agents, binders, lubricants and disintegrating agents.

Examples of disintegrants include starch and starch derivatives, and other swellable polymers, for example cross-linked polymeric disintegrants such as cross-linked carboxymethylcellulose, cross-linked polyvinylpyrrolidone and starch glycolates.

Examples of lubricants include stearates such magnesium stearate and stearic acid. lactose, and sugar alcohols such as xylitol, sorbitol and mannitol; or modified cellulose or cellulose derivative such as powdered cellulose or microcrystalline cellulose or carboxymethyl cellulose.

Examples of binders and granulating agents include polyvinylpyrollidone. Where the diluent is not naturally very sweet, a sweetener can be added, for example ammonium glycyrrhizinate or an artificial sweetener such as aspartame, or sodium saccharinate.

A capsule composition typically will comprise an outer shell or casing which may, for example, be formed from hard or soft forms of gelatin or gelatin-equivalents in conventional fashion. The outer shell is filled with microspheres containing a biologically active substance as hereinbefore defined and optionally one or more auxiliary agents such as pH controlling agents/buffering and diluents such as the sugars and sugar alcohols listed above.

The microspheres can be agglomerated into granules prior to tableting or filling into a capsule and, as such can be wet granulated or dry granulated as desired.

When the capsule filling is in liquid or semi-solid form, the microspheres can be suspended in a semi-solid carrier material such as a polyethylene glycol or a liquid carrier such as a glycol, e.g. propylene glycol, or glycerol, or a vegetable or fish oil, for example an oil selected from olive oil, sunflower oil, safflower oil, evening primrose oil, soya oil, cod liver oil, herring oil, etc. In general, it is preferred that the capsule is in solid or semi-solid form when hard gelatin capsules are used; liquid or semi-solid forms being preferred with soft gelatin capsules.

Tonicity adjusting agents such as sodium chloride, or sugars, can be added to provide an environment of a particular osmotic strength, for example isotonicity. One or more pH adjusting agents, such as buffering agents can also be used to adjust the pH to a particular value, and preferably maintain it at that value. Examples of buffering agents include sodium citrate/citric acid buffers and phosphate buffers.

Compositions of the invention can be provided in a powder form for reconstitution as a solution. As such they can also contain soluble excipients such as sugars, buffering agents such as citrate and phosphate buffers, and effervescent agents formed from carbonates, e.g bicarbonates such as sodium or ammonium bicarbonate, and a solid acid, for example citric acid or an acid citrate salt.

The compositions can be formulated to delay release of the microcapsules until the composition has reached the stomach, or has passed through the stomach into the duodenum, ileum or colon. Thus, for example, the compositions can be presented in enteric-release or colonic release formulations. Such formulations can be in the form of capsules or tablets coated with or comprising a release retarding agent that delays disintegration and release of the microcapsules until a particular set of conditions (e.g. pH) are encountered. Such release retarding agents are well known per se and need not be discussed in detail herein.

The compositions of the invention can be used in methods of treatment or therapy, or methods of immunisation, for example. The amount of the composition administered, and in particular the amount of biologically active ingredient administered to a patient per day will depend upon the potency of the active substance the particular condition or disease under treatment and its severity, and ultimately it will be at the discretion of the physician. The amount administered however will typically be a non-toxic amount effective to treat the condition in question, or effective to produce the desired effect (e.g. immune response).

For example, the amount of active substance administered to a patient may be from 0.0001 to 0.1, preferably 0.001 to 0.05 gram per kilogram body weight, with quantities towards the lower end of the range being administered when the active substance is a vaccine substance such as an antigen or DNA.

Where the compositions are non-vaccine compositions, they can be administered in single or multiple dosage units per day, for example from one to four times daily, preferably one or two times daily. With vaccine compositions, immunisation may be effected by means of a single administration, but more usually will be followed up by one or more booster administrations.

Although a major use envisaged for the crosslinked hemicellulose microparticles of the invention is as a delivery system for pharmaceutical agents such as drugs, vaccines and diagnostic agents, including such agents for use in a veterinary as well as human context, the microparticles of the invention are believed to be useful in other fields, for example as vehicles for the delivery of pesticides, herbicides, plant growth hormones, nutrients, trace elements and other agricultural and horticultural substances.

### BRIEF DESCRIPTION OF THE DRAWINGS

FIG. 1 is a graph showing the particle size distribution of a gelled 2% solution of arabinoxylan after freeze drying, ball milling in a Cyclotech 1093 sample mill, and dispersion in de-ionised water.

FIG. 2 illustrates the particle size distribution of the sample shown in FIG. 1 after a total of 8 hours ball milling.

FIG. 3 is a graph illustrating the particle size distribution in the dry unhydrated state of the gelled AXF of FIG. 1 after ball milling for 8 hours.

FIG. 4 is a graph illustrating the particle size distribution in the dry unhydrated state of the gelled AXF of FIG. 1 after ball milling for 17.25 hours.

FIG. 5 shows the particle size distribution of the AXF of FIG. 4 after hydration.

FIG. 6 shows the particle size distribution of the AXF after ball milling for 24 hours and dispersion in de-ionised water.

FIG. 7 shows the particle size distribution of microparticles of crosslinked AXF comminuted by vortexing or sonicating.

FIGS. 8a to 8d show the immune responses to the test antigen ovalbumen (OVA) formulated in uncrosslinked AXF;

FIG. 9 shows the immune responses to OVA or a DNA formulated with crosslinked AXF.

FIG. 10 shows the mean endpoint titres for immune responses to OVA or a DNA formulated with a crosslinked AXF.

FIG. 11 illustrates the immune responses to DNA formulated with uncrosslinked AXF.

### DETAILED DESCRIPTION OF THE PREFERRED EMBODIMENTS

The invention will now be described by reference to the following examples which are purely exemplary and which do not limit the scope of the invention in any way.

### EXAMPLE 1

Preparation of Cross-linked Arabinoxylan ferulate (AXF) Microspheres

In this example and the following examples, the AXF was extracted from maize bran and purified by an alkali/alcohol extraction method substantially as described in WO-A-93/10158.

A 2% solution of AXF (1 litre) was autoclaved at 126° C. for 11 minutes to remove nuclease activity and was then gelled by the addition of 3% hydrogen peroxide (1 ml) and horseradish peroxidase (1 ml at a concentration of 1 mg/ml). The mixture was left to gel overnight and was then transferred into metal trays and frozen. The gel was freeze-dried for a period of four days after which time it had formed into straw-coloured brittle mats. This material was broken into small pieces and milled using either a Cyclotech 1093 sample mill or a combination of the Cyclotech mill and a Pascal Ball Mill. The sizes of the particles of gelled AXF following these treatments were determined using a Coulter particle size analyser. Dry particles were analysed using a dry powder module. In order to analyse hydrated particles, a sample was dispersed in de-ionised water and tested using a small volume module.

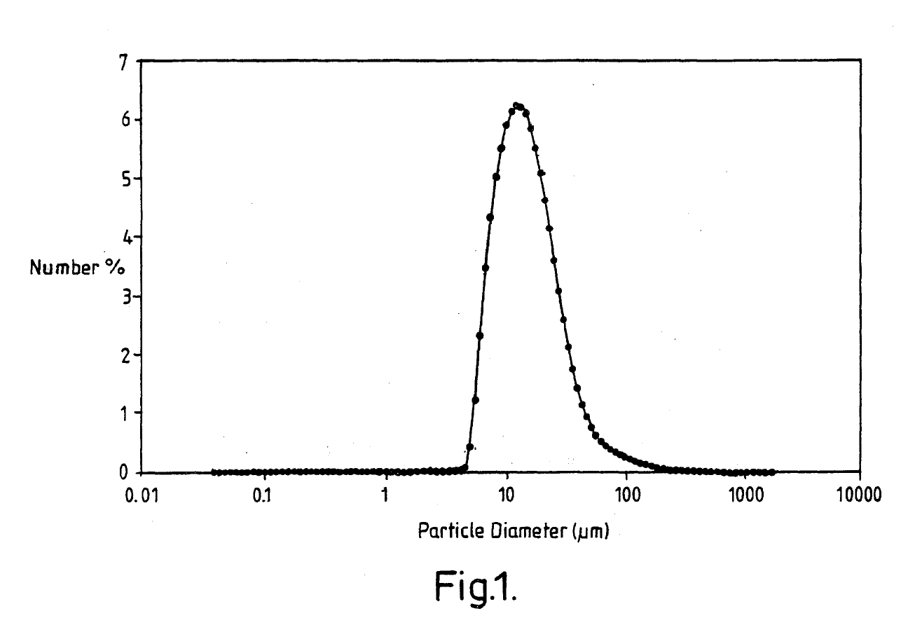

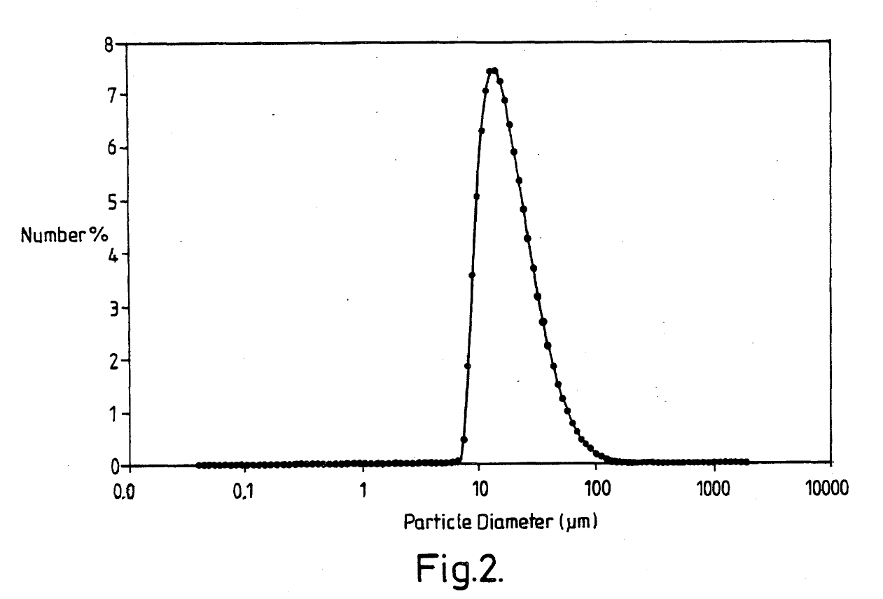

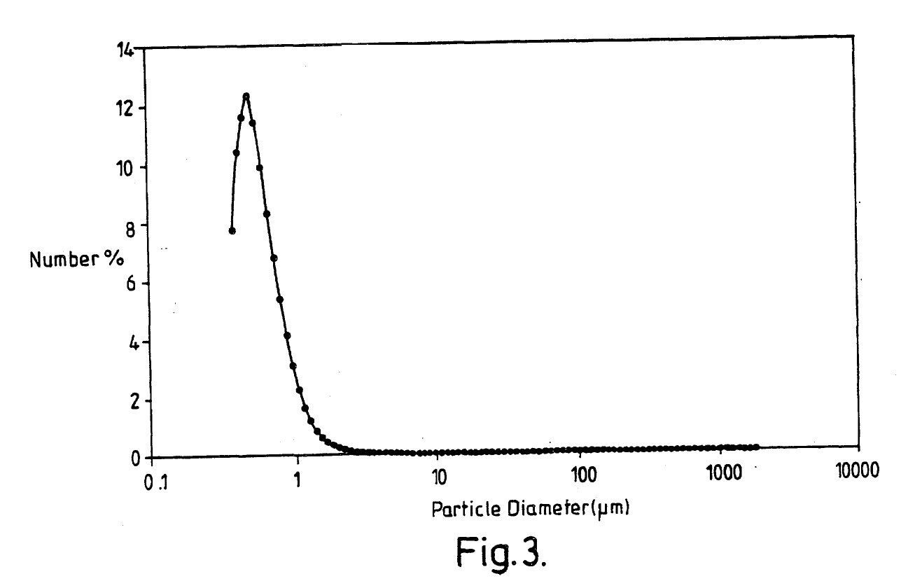

The mean particle size of hydrated pre-gelled AXF particles following cyclone milling is shown in FIG. 1. An average of three runs was taken and this indicated a mean particle size of 21.04 μm. In order to decrease the particle size further, the AXF particles were ball milled for a total of 8 hours and were then analysed after hydration (FIG. 2). The data suggest that the particle size did not change substantially (23.01 μm compared to 21.04 μm) but visual inspection of the material suggested otherwise. It was decided to run a sample of the milled material as a dry powder to eliminate the possibility that the particles had aggregated in some way. The result from an average of three runs, shown in FIG. 3, gave an average particle size of approximately 0.6 μm.

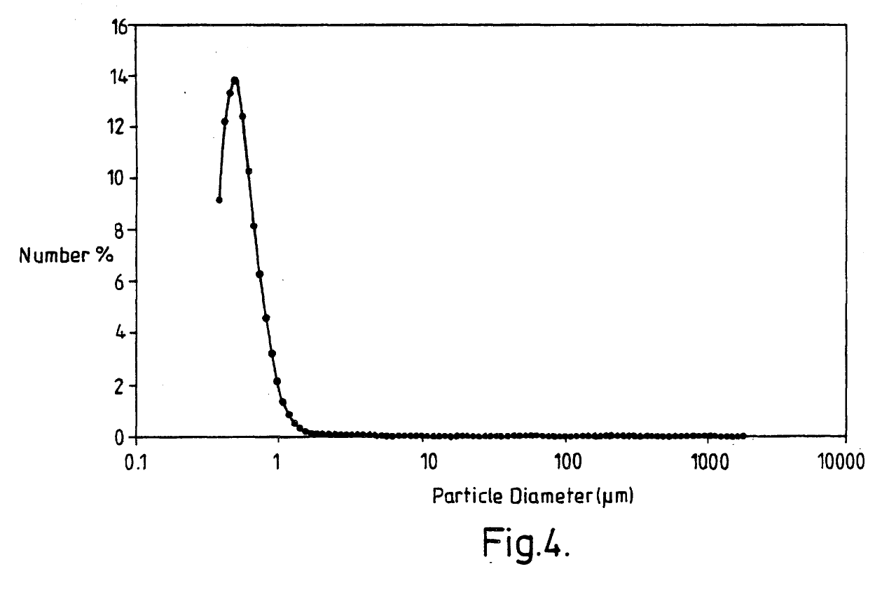

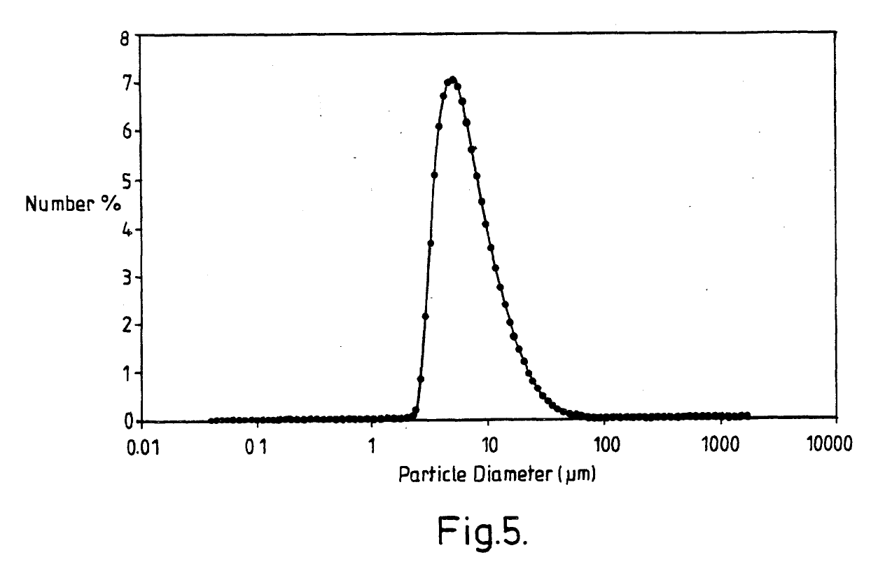

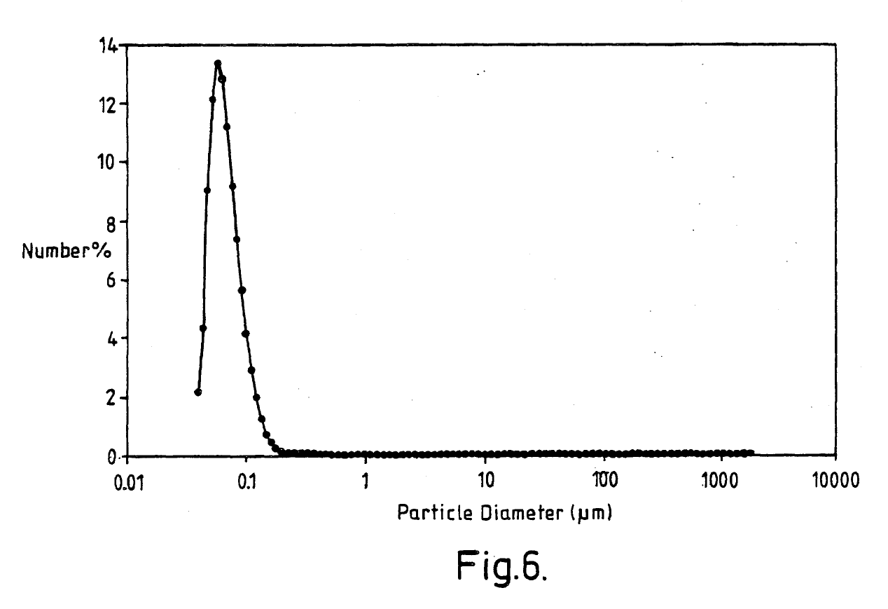

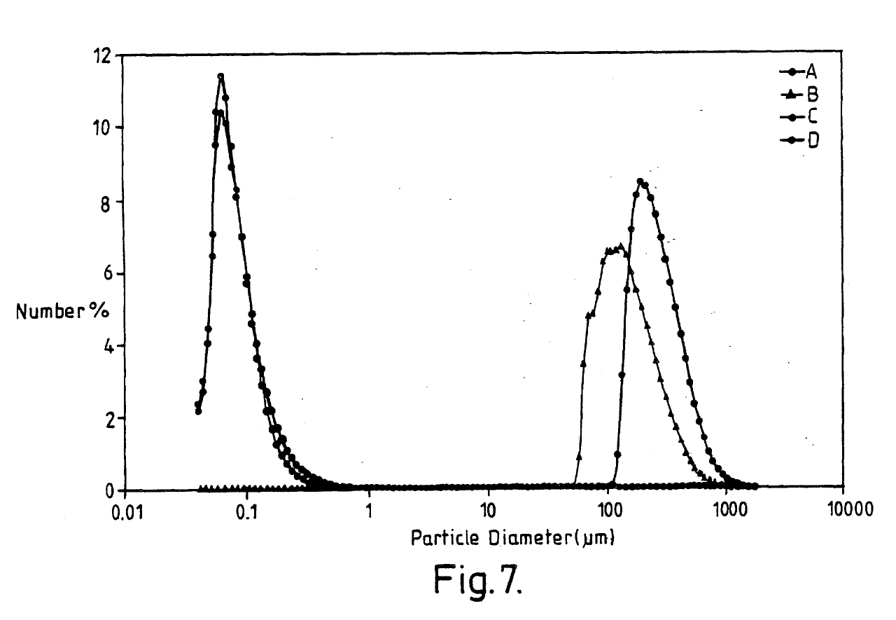

In the foregoing tests, hydration of the microparticles following milling gave particle size ranges somewhat larger than the particle sizes given by measurement of the dry microparticulates. In an attempt to ascertain if the particles aggregated in water, the particles were dispersed in both water and ethanol, stirred for 10 minutes, and were then viewed by light microscopy. Microscopic analysis revealed that the AXF microparticles swell, rather than aggregate, when dispersed in water. It is possible that ball milling resulted in the compaction of AXF particles rather than a reduction in particle size: thus, following hydration, the particle size remained constant. However, further milling (an additional 8.25 hours) did produce a reduction in the mean particle size of both the dry powder and hydrated microparticles, shown in FIGS. 4 and 5, respectively. An additional 7.75 hours of milling (to make a total of 24 hours) resulted in hydrated particles with a mean particle size of 0.0769 μm (FIG. 6).

Microparticles of the type described above may be used to form dry powder formulations, for example nasal sprays. Such dry powder formulations may be used to deliver DNA, for example DNA vaccines or DNA therapeutic agents. The DNA (or other therapeutic agent) can be incorporated into the microparticles during the cross-linking reaction, or they can be incorporated during hydration of the dry milled microparticles by soaking the dry particles in a solution containing the therapeutic agent so that the therapeutic agent is taken up by the microparticles. The latter approach permits greater flexibility in that the microparticulate delivery vehicle can be provided as a powder and added to a solution of a therapeutic agent such as a DNA (e.g. a DNA vaccine) vaccine prior to administration (e.g. by injection).

### EXAMPLE 2

Preparation of Crosslinked AXF Particles by Vortexing or Sonication

It was found that by mixing AXF with the crosslinking reagents (see Example 1) in a bijoux tube, followed quickly by drawing the mixture into a syringe, that successful crosslinking of AXF occurred within the syringe. However, the resulting gel could only be readily ejected from the syringe through a 19 GA needle. If crosslinking was performed on an AXF solution of less than 4% the resultant gel lost integrity when extruded. The 4% gels retained their integrity, however, and this gel strength was used for further investigation. Subcutaneous injection of a 4% gel made in this manner resulted in an inert bleb that neither increased nor decreased in size over a number of weeks, nor resulted in any pathology. This demonstrated that the crosslinked AXF is biocompatible and hence that it should be suitable for in vivo use.

Routine injection during immunisation experiments is performed through much finer needles (27 GA) than those used above. AXF crosslinked within a syringe cannot be ejected through such a narrow needle. However, it was found that ejecting the crosslinked AXF through a 27 GA needle into buffer (PBS) followed by a brief period (10 seconds) of vortexing broke the crosslinked AXF up such that it could be drawn into a syringe and ejected through a 27 GA needle.

AXF was prepared within a syringe as described above but, instead of vortexing, sonication was performed at an amplitude of 105 μm for varying times in a Soniprep 150 disruptor (Sanyo).

The sizes of particles generated by vortexing and sonication were analysed by Coulter counter particle analyser and the results are set out in Table 1 below. In each of the experiments, 1 ml of 4% AXF in phosphate buffered saline was crosslinked in a syringe overnight and then ejected into 1 ml 1×PBS in a bijoux tube thorough a 19 GA needle prior to the vortexing or sonication step. As can be seen from the results in the Table, increasing the sonication time decreased the size of particles generated. However, all of the preparations could be ejected through a 27 GA needle.

TABLE 1

| Treatment | Mean Particle Size Diameter (μm) |
|---|---|
| Vortex, 10 seconds | 308 |
| Sonicate, 5 seconds | 177 |
| sonicate, 10 seconds | 0.100 |
| sonicate, 30 seconds | 0.0929 |

### EXAMPLE 3

The use of Uncrosslinked AXF as a Vaccine Delivery Vehicle

Determination of the Immunological Properties of AXF

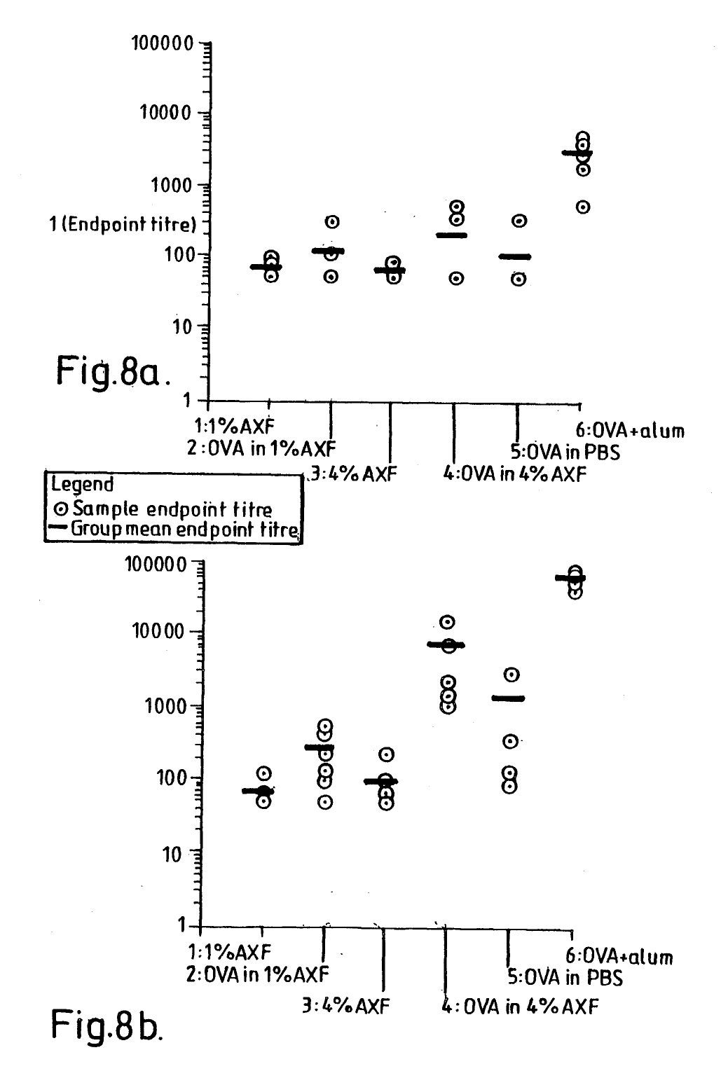

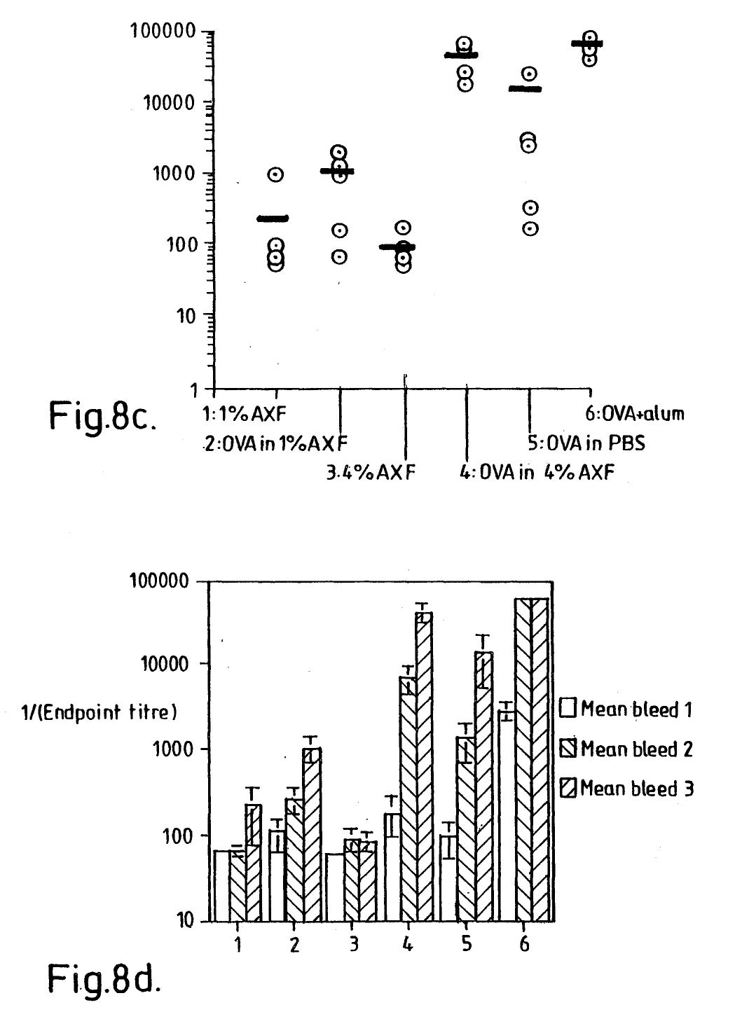

The immune response to a standard test protein, ovalbumin (OVA) was compared when mice were immunised subcutaneously with OVA delivered in either a physiological buffer (phosphate buffered saline, PBS), or a solution containing AXF, or complexed with the adjuvant alum. The immunisation regime and doses are described are set out below in Tables 2 and 3. The total anti-OVA IgG antibody titres were determined by a standard ELISA method and are displayed in FIGS. 8a-d. All statistical comparisons between endpoint titres were assessed by Mann-Whitney U-test at the 5% level of significance.

TABLE 2
Immunisation Regime

| Event | Day |
|---|---|
| Primary immunisation | 0 |
| 1^{st} bleed | 22 |
| 1^{st} boost | 29 |
| 2^{nd} bleed | 53 |
| 2^{nd} boost | 64 |
| 3^{rd} bleed | 85 |

TABLE 3
Groups, Formulations and Routes

| Group | Formulation | Route |
|---|---|---|
| 1 | 1% AXF | s/c |
| 2 | 1% AXF + OVA | s/c |
| 3 | 4% AXF | s/c |
| 4 | 4% AXF + OVA | s/c |
| 5 | OVA | s/c |
| 6 | OVA + alum | s/c |

As can be seen from FIGS. 8a-8d, at 22 days after the primary immunisation, the response from the OVA+alum control group (number 6) was significantly higher all the other groups. There was no statistically significant difference between the non-alum groups. However, the mean endpoint titres of the OVA+AXF groups (OVA+1% AXF, group 2, mean 106; OVA+4% AXF, group 4, mean 180) were somewhat higher than the OVA+PBS group (group 5, mean 97) suggesting that an increase may be observed post-boost.

At 24 days after the first boost, the response from the group immunised with OVA in 4% AXF showed a greater response than that immunised with OVA in PBS (Group 4>Group 5, P=0.041). In contrast, the group immunised with OVA in 1% AXF did not show a greater response than the PBS control group (Group 2>Group>5, P=0.155). The alum group still responded better than the 4% AXF group (Group 6>Group 4, P=0.001). This indicated that AXF appeared to possess adjuvant activity, though not as intense as that of alum. To determine whether this activity could be boosted further, a third immunisation was performed 11 days after the second boost.

At 21 days after the second boost, there was no significant difference between the OVA+alum and OVA+4% AXF groups (Group 6>Group 4, P=0.155). There was still no difference between the OVA+1% AXF and OVA+PBS control group (Group 5>Group 2, PF=O. 123).

The results demonstrate that uncrosslinked AXF (at a concentration of 4%) possesses adjuvant activity comparable to that of alum after two boost immunisations.

### EXAMPLE 4

The use of Crosslinked AXF as a Vaccine Delivery Vehicle

Determination of the Immunological Properties of Crosslinked AXF

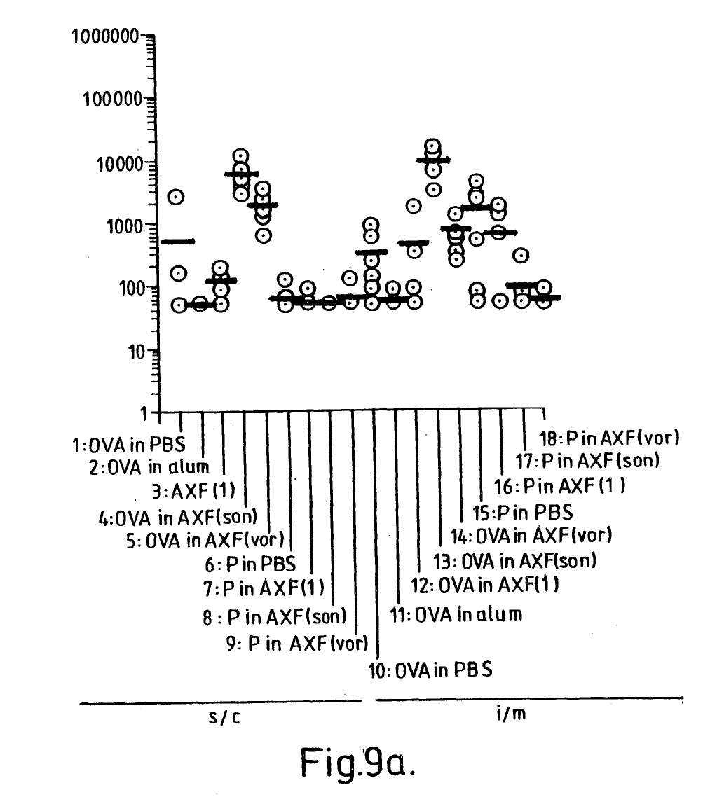

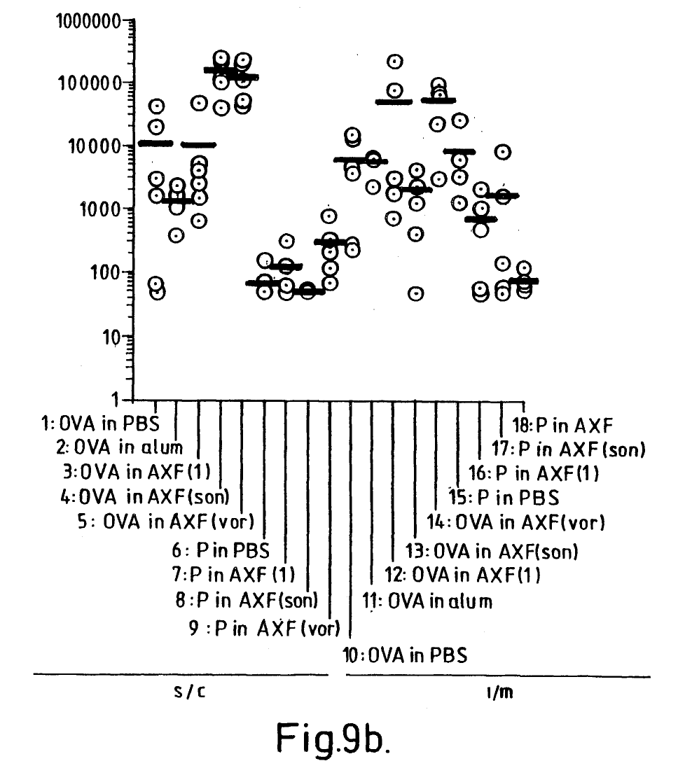

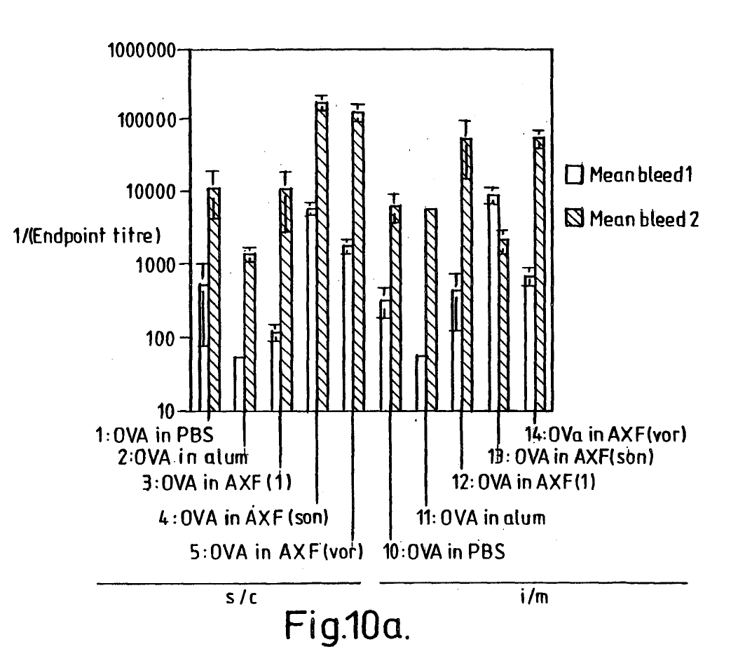

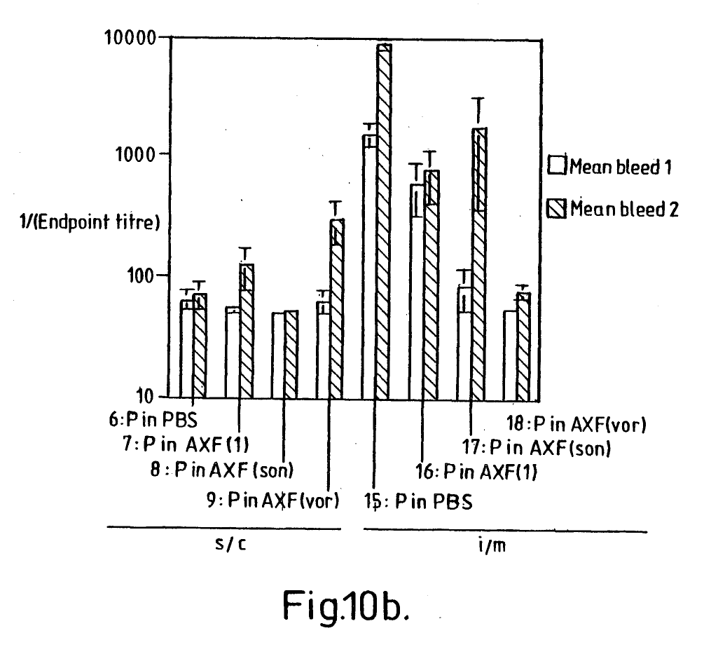

The immune responses of mice to the test antigen OVA were compared between protein administered in PBS, alum, 4% AXF solution and crosslinked AXF (either sonicated or vortexed). Immunisations were performed via both the subcutaneous (s/c) and intramuscular i/m) routes. For s/c immunisation, 100 μg of OVA was administered in a 300 μl single dose. For i/m immunisation, 100 μg of OVA was administered as 2×150 μl doses into the quadriceps. The immunisation regime and doses are described in Tables 4 and 5 below. The total anti-OVA IgG antibody titres were determined by ELISA and are displayed in FIGS. 9 and 10.

The first bleed was performed 21 days after the primary immunisation. Surprisingly, the response shown by the animals immunised with OVA+alum (both s/c or i/m) was much lower than that observed in Example 3; indeed they were only just above background. Possibly this was due to extended incubation of the protein with alum (conducted to ensure that each formulation was treated identically). Comparison with the OVA+PBS groups and the results from Experiment 1 are informative however.

The response from the groups immunised with OVA in uncrosslinked AXF showed no difference to those immunised with OVA in PBS (Group 3>Group I;P=0.35 and Group 10>Group 12;P=0.396). This is consistent with the results from Example 3 and extends the results of the previously described protein immunisations to the intramuscular route.

Subcutaneous delivery of OVA formulated in crosslinked AXF that had been either sonicated or vortexed resulted in a significantly higher response than delivery in either PBS or uncrosslinked AXF (Group 4>Group 1, P=0.001; Group 4>Group 3, P=0.001; Group 5>Group 1, P=-0.021; Group 5>Group 3, P=0.001) i.e. crosslinked AXF is showing adjuvant activity. Additionally the sonicated group showed a higher response to the vortexed one (Group 4>Group 5, P=0.001), suggesting that variation in particle size can modulate the magnitude of the immune response (with smaller particles eliciting a greater response in this case. While statistical comparisons between the results of this experiment and Example 3 cannot be conducted, it is promising that the mean endpoint titre from the vortexed group 5 (mean 5864) is greater than the OVA+alum group from Example 3 (mean 2870). This suggests that the adjuvant activity of crosslinked AXF may be comparable to that of alum.

In the case of intramuscular immunisation, sonicated AXF also significantly increased the immune response compared to delivery in PBS or uncrosslinked AXF (Group 13>Group 10, P=0.001; Group 13>Group 12, P=0.002). However formulation of OVA in vortexed crosslinked AXF did not result in a significantly higher response than formulation in either PBS or uncrosslinked AXF (Group 14>Group 10, P=0.066; Group 14>Group 12, P=0.210). The lower response to the vortexed particles mirrors the subcutaneous case.

Interestingly the response to immunisation with OVA formulated in PBS or sonicated crosslinked AXF is route-independent (Group 10>Group 1, P>0.155; Group 13>Group 4, P=0.197), while subcutaneous immunisation with OVA formulated in vortexed crosslinked AXF gave a greater response than intramuscular (Group 5>Group 14, P=0.008). Thus there is an observable difference in the immune response to antigen when delivered in crosslinked AXF according to the size of the AXF particles in the preparation and the route of delivery.

At 20 day post-boost, the response to subcutaneous immunisation with crosslinked AXF was higher than uncrosslinked AXF or PBS (Group 4>Group 3, P=0.002; Group 5>Group 3, P=0.002; Group 4>Group 1, P=0.002; Group 5>Group 1, P=0.001), consistent with the results from bleed 1. However there was no difference between the groups that received sonicated and vortexed preparations (Group 4>Group 5, P=0.242). Comparison of mean titres with those from Example 3 again indicates that crosslinked AXF has adjuvant activity comparable to alum when delivered subcutaneously (compare OVA+alum Example 3 mean=62200, OVA+AXF sonicate Example 4 mean=165000 and OVA+AXF vortex Experiment H mean=122000).

In the case of the intramuscular groups, much larger intra-group variation is apparent from the second bleed. This may be due to the practical difficulty of injecting the required volume into the target muscle. Most strikingly, in contrast to the first bleed and the subcutaneous second bleed, the intramuscular vortexed group shows a greater response than the sonicated (Group 14>Group 13, P=0.004) which itself shows no difference to formulation in liquid AXF (Group 12>Group 13, P>0.294). Indeed the response in the sonicated group is in fact lower at bleed 2 compared to bleed 1 (mean bleed 1=8850, mean bleed 2=2120).

Thus, the experiments demonstrate that subcutaneous immunisation with antigen formulated in crosslinked AXF produces an immune response greater than uncrosslinked AXF and comparable to, if not greater than, formulation with alum. The size of the crosslinked AXF particles may be important for the timing of the immune response, formulation with larger particles requiring a boost immunisation to make the response equivalent to that achieved with the smaller particles. Intramuscular delivery shows more variation in the magnitude of the immune response.

The low endpoint titres in the groups that received antigen complexed with alum prevents a statistical comparison of the magnitude of the adjuvant effect of AXF with this commercial adjuvant.

In the foregoing Example, for technical reasons the total amount of AXF administered in each dose was lower in the crosslinked samples compared to the uncrosslinked ones. The results shown in Example 3 demonstrated that increasing the AXF dose increased the adjuvanticity of AXF. If the adjuvant activity of the crosslinked samples was purely due to the amount of AXF in each dose it would therefore be predicted that the crosslinked samples would show a lower response than the uncrosslinked samples. In fact the opposite was observed. Therefore the increased adjuvanticity of the crosslinked samples as compared to the uncrosslinked samples in Example 4 must be due to the crosslinking process.

TABLE 4
Immunisation Regime

| Event | Day |
|---|---|
| Primary immunisation | 0 |
| 1^{st} bleed | 21 |
| Boost Immunisation | 30 |
| 2^{nd} bleed | 50 |

TABLE 5
Groups, Formulations and Routes

| Group | Route | Formulation |
|---|---|---|
| 1 | s/c | OVA in PBS |
| 2 | s/c | OVA in alum |
| 3 | s/c | OVA + AXF (1) |
| 4 | s/c | OVA + AXF (son) |
| 5 | s/c | OVA + AXF (vor) |
| 6 | s/c | P in PBS |
| 7 | s/c | P + AXF (1) |
| 8 | s/c | P + AXF (son) |
| 9 | s/c | P + AXF (vor) |
| 10 | i/m (quad) | OVA in PBS |
| 11 | i/m (quad) | OVA in alum |
| 12 | i/m (quad) | OVA + AXF (1) |
| 13 | i/m (quad) | OVA + AXF (son) |
| 14 | i/m (quad) | OVA + AXF (var) |
| 15 | i/m (t.a.) | P in PBS |
| 16 | i/m (t.a.) | P + AXF (1) |
| 17 | i/m (t.a.) | P + AXF (son) |
| 18 | i/m (t.a.) | P + AXF (vor) |

Key: AXF (1) indicates biomolecule in 4% AXF solution, AXF (son) and AXF (vor) indicate biomolecule incorporated into crosslinked AXF followed by either sonication or vortexing.

### EXAMPLE 5

DNA Delivery in vitro

The Use of AXF as a DNA Delivery Vehicle

In order to determine whether AXF can modulate the ability of DNA to transfect mammalian cell lines, Chinese Hamster Ovary (CHO) cells were exposed to the pSV-β-galactosidase plasmid (Promega) in the presence of the LIPOFECTAMINEPLUS™ transfection reagent or AXF. Transfection efficiency was assessed by determining the β-galactosidase activity of lysates of the treated cells using a β-galactosidase assay kit (Promega) and the BCA Protein Assay Kit available from Bio-Rad. The results of the assays are shown in Table 6 below.

TABLE 6
Rates of Transfection of Chinese Hamster Ovary (CHO) Cells by the
pSV-β-galactosidase plasmid (Promega) Expressed as Level of
(β-galactosidase Activity per mg of Total Protein in Cell Lysates

| Sample | β-galactosidase activity per mg of protein |
|---|---|
| CHO | 1 |
| CHO + DNA | 2.5 |
| CHO + DNA + | 343 |
| LIPOFECTAMINEPLUS ™ | |
| CHO + DNA + 0.2% AXF | 2.60 |
| CHO + DNA + 2% AXF | 2.00 |
| CHO + DNA + 6% AXF | 3.20 |

As can be seen from the results in Table 6, there was no significant difference between the β-galactosidase activity of the cells exposed to DNA on its own or in the presence of varying concentrations of AXF. In contrast, the use of the LIPOFECTAMINEPLUS™ reagent (GibcoBRL) resulted in β-galactosidase activity approximately two orders of magnitude higher. The results thus demonstrate that, uncrosslinked AXF does not promote uptake of DNA by CHO cells.

### EXAMPLE 6

DNA Deliver in vivo

DNA Immunisation using Uncrosslinked AXF as the Delivery Vehicle

Intramuscular injection of microgram quantities of plasmid DNA coding for antigenic proteins has been shown to elicit an immune response to the encoded protein. We have previously shown in the laboratory that intramuscular injection of 10-100 μg of a plasmid (P) carrying a gene coding for an antigenic protein (A) from a pathogenic bacterium under control of eukaryotic expression signals leads to the development of anti-A antibodies in the test organism. In order to determine whether formulation of DNA in AXF can modulate the immune response, immunisation experiments utilising uncrosslinked AXF were carried out in a manner analogous to the experiments described in Examples 3 and 4.

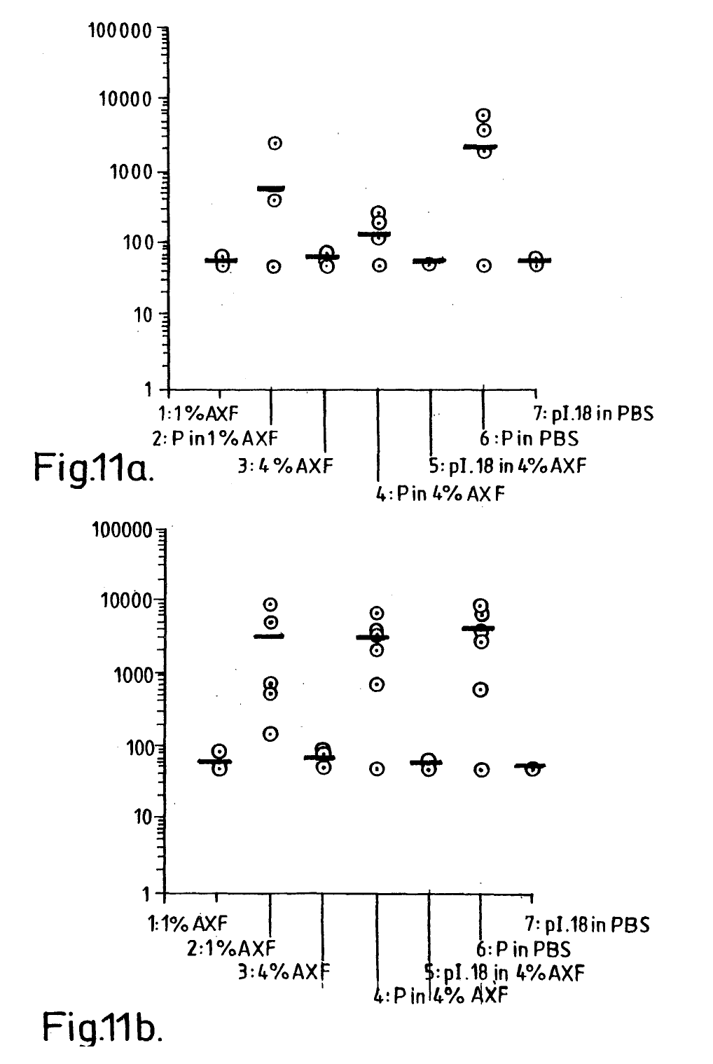

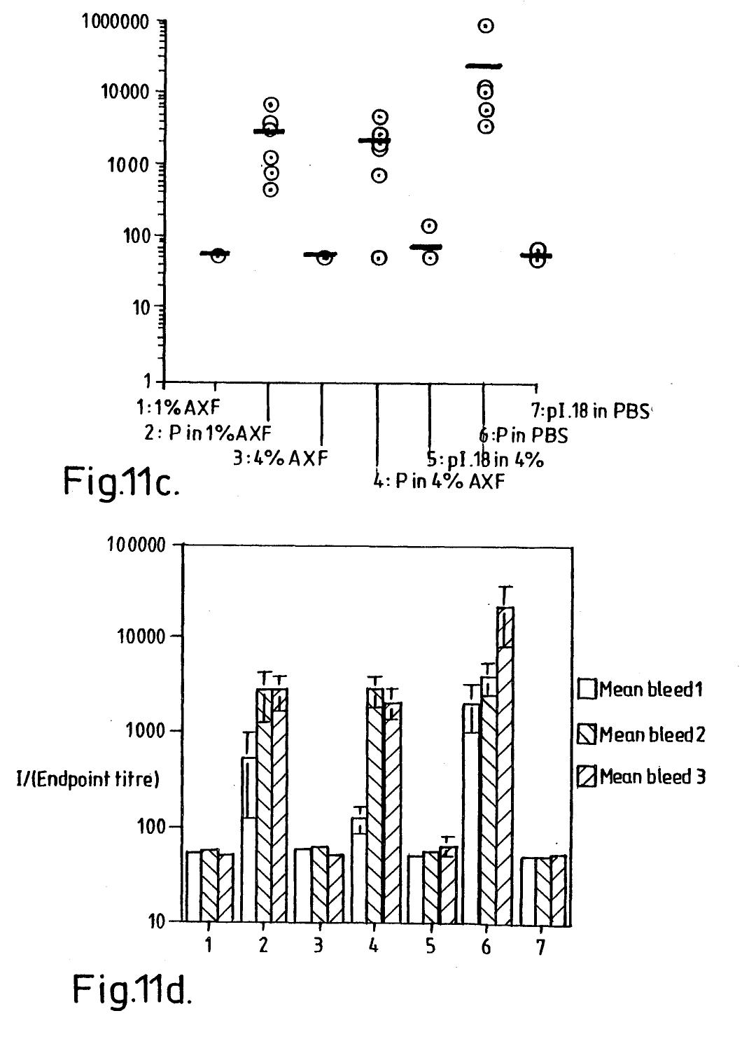

Thus, the immune response to antigenic protein A was compared when mice were immunised with a test plasmid P (coding for protein A) delivered in a variety of formulations, either PBS or varying concentrations of AXF. The immunisation regime and doses were as set out in Tables 7 and 8. The DNA was administered intramuscularly in a dose of 100 μl containing 20 μg of plasmid DNA. The total anti-A IgG antibody titres were determined in standard fashion by ELISA and the results are displayed in FIG. 11 and Table 9.

TABLE 7
Immunisation Regime

| Event | Day |
|---|---|
| Primary immunisation | 0 |
| 1^{st} bleed | 22 |
| 1^{st} boost | 29 |
| 2^{nd} bleed | 53 |
| 2^{nd} boost | 64 |
| 3^{rd} bleed | 85 |

TABLE 8
Groups, Formulations and Routes

| Group | Formulation | Route |
|---|---|---|
| 1 | 1% AXF | i/m |
| 2 | 1% AXF + P | i/m |
| 3 | 4% AXF | i/m |
| 4 | 4% AXF + P | i/m |
| 5 | 4% AXF + pI.18 | i/m |
| 6 | P in PBS | i/m |
| 7 | pI.18 in PBS | i/m |

TABLE 9
P-Values as Calculated for Mann-Whitney Comparison Between Indicated
Groups

| Comparison (group | P-Value | | |
|---|---|---|---|
| numbers) | Bleed 1 | Bleed 2 | Bleed 3 |
| [P + 1% AXF] > [pI.18] | 0.294 | 0.001 | 0.001 |
| (group 2 > 7) | | | |
| [P + 4% AXF] > [pI.18] | >0.120 | 0.008 | 0.013 |
| (group 4 > 7) | | | |
| [P + PBS] > [pI.18] | >0.120 | 0.008 | 0.001 |
| (group 6 > 7) | | | |
| [P + PBS] > [P + 1% | 0.242 | 0.350 | 0.043 |
| AXF]] (group 6 > 2) | | | |
| [P + PBS] > [P + 4% | >0.242 | >0.35 | 0.002 |
| AXF]] (group 6 > 4) | | | |

As can be seen from the results in Table 9, there was no difference in specific IgG titres between the groups given plasmid in PBS, 1% or 4% AXF either 22 days post-primary or 24 days post-first boost immunisation. At 22 days post-primary immunisation there was not a significant difference between any of the test groups and the group immunised with the control plasmid pI.1 8 which was identical to P except for the absence of the gene coding for protein A. By the second bleed, all the test groups showed a significantly higher specific IgG titres than the control pI.1 8 group. At 21 days post-second boost, the AXF-formulated groups showed a lower response than the control PBS-formulated group, although taking into account the considerable overlap in titres between groups (see FIG. 11) it is possible that this is due to experimental variation rather than a suppression of the immune response to proteins coded for by injected DNA.

The results demonstrate that AXF does not have a large effect, either stimulatory or inhibitory, on the immunogenicity of plasmids coding for antigenic proteins.

### EXAMPLE 7

DNA Immunisation using Crosslinked AXF as the Delivery Vehicle

The kinetics of the immune response to genes coding for antigenic proteins administered on a plasmid can be modulated by controlled release of the plasmid from the delivery medium. Incorporation of DNA into AXF gels has the potential to result in a delayed release of plasmid in vivo. This experiment was designed to determine whether incorporation of an antigen-encoding plasmid into crosslinked AXF affects the immune response to the antigen. Generation of an immune response to a protein encoded on a plasmid has not been observed when the plasmid was administered subcutaneously. To determine whether plasmid incorporation into AXF can elicit an immune response by this route, mice were immunised both intramuscularly (i/m) and subcutaneously (s/c).

The immune response to protein A was compared when mice were immunised with plasmid P delivered in either PBS, 4% AXF solution or crosslinked AXF sonicated or vortexed. The immunisation regime and doses are described in Tables 4 and 5 above. The total anti-A IgG antibody titres were determined by ELISA and are displayed in FIGS. 9 and 10.

At 21 days after the primary immunisation, the intramuscular results were consistent with previous experiments in that formulation in uncrosslinked AXF showed no significant difference to plasmid delivered in PBS (see Table 9). In contrast, incorporation into crosslinked AXF followed by either sonication or vortexing resulted in a lower response than formulation in PBS. After the boost, all the AXF formulated groups, including the uncrosslinked AXF, showed a lower response than P delivered in PBS. These results may indicate that crosslinked AXF as formulated in these experiments sequesters DNA too tightly to elicit strong immune responses. Alternatively the large delivery volumes required in these experiments may not have been optimal for intramuscular delivery of plasmid, resulting in reduced expression of the antigenic protein

In the case of the subcutaneous immunisations, at 21 days post-primary immunisation, no seroconversion was observed in any of the samples, as may be expected from the previous observations. More interestingly, at 20 days post-boost, specific antibody titres from all the animals immunised with vortexed AXF containing plasmid P had increased, possibly indicating an AXF-mediated immune response via the subcutaneous route.

### EXAMPLE 8

Determination of the Ability of Injectable Crosslinked AXF to Incorporate Biomolecules

8A. Incorporation of DNA

In order to determine whether the injectable crosslinked AXF described in the previous examples can entrap DNA, the standard plasmid pUC18 was incorporated into an AXF matrix by crosslinking for either 15 minutes or overnight as described above. The resulting crosslinked gel was then extruded through a 19 GA needle into buffer and vortexed. The concentration of pUC18 in the resultant supernatant was assessed by electrophoresis immediately, and after one or two overnight incubations. In all cases, the AXF used was rendered nuclease free prior to the experiment. The concentration of plasmid DNA in the supernatants immediately after introduction into buffer was much lower (approximately 10%) than the control samples that were uncrosslinked, thus indicating that the plasmid was successfully incorporated into the matrix. After one overnight incubation, plasmid concentration in the supernatant increased demonstrating that over time the incorporated DNA was released. The length of time allowed for crosslinking evidently affected the rate of release; the sample crosslinked for 15 minutes had released all its encapsulated DNA after one overnight incubation, compared to approximately 30% for the crosslinked overnight sample. After two overnight incubations all the DNA had been released from the AXF that was crosslinked overnight.

The results of this study thus indicate that DNA can be formulated in crosslinked AXF such that it is injectable and demonstrates time-delayed release in vitro.

8B. Incorporation of Protein

In order to determine whether the injectable crosslinked AXF can entrap protein, an experiment equivalent to the DNA experiment described above in Example 8A was conducted incorporating OVA into the AXF gel. The concentration of OVA in the supernatant was assessed by Western blotting (Western blot not shown). There were no detectable differences in OVA concentration between the control and test samples indicating that OVA was not entrapped in the matrix. This result was confirmed by a semi-quantitative Western slot blot method (not shown). Possibly the difference in entrapment between the DNA above and OVA was due to the difference in size between the molecules. OVA has a molecular weight of 43 kDa compared to approximately 2 MDa for the 3 kbp pUCI8 plasmid. It is believed that the smaller molecule may migrate through the AXF matrix more readily.

The results thus illustrate that the test protein ovalbumin could not be sequestered by crosslinked AXF and hence the adjuvant effect of the AXF cannot be ascribed to sequestration.

### EXAMPLE 9

DNA Immunisation by Co-administration of Crosslinked AXF Microspheres and Plasmid

Encoding a Vaccine Antigen

In order to determine whether AXF microspheres can exert an adjuvant effect when co-administered with DNA encoding a vaccine antigen, rather than being formulated such that the DNA is encapsulated within the AXF microspheres, the following experimental protocol is followed.

9A. Preparation of Immunogens

Preparation of AXF Solution

AXF powder is dissolved in sterile, endotoxin-free PBS (Sigma) to a concentration of 6% w/v with stirring overnight at room temperature. The solution is autoclaved and any undissolved matter removed by centrifugation at 6,000 rpm for 10 minutes in a Sorvall RC5B Refrigerated Superspeed Centrifuge. The supernatant is decanted and used immediately.

Preparation of AXF Gel

AXF gel is made in 1.5 ml aliquots in sterile flat-bottomed tubes. 1.5 ml 6% AXF solution is mixed with 3 μl 1 mg/ml horseradish peroxidase (Sigma) and gently shaken for a few seconds. 30 μl 0.3% v/v hydrogen peroxide is added and the solution immediately mixed twice by taking up into and expelling from a sterile 2 ml syringe. After two mixing cycles the solution is drawn up into the same syringe and left to set overnight on the bench.

Preparation of AXF Sonicate

Each AXF aliquot is extruded through a 19 GA syringe into a 1.5 ml aliquot of sterile, endotoxin-free PBS in sterile, flat-bottomed tubes with a diameter of 1 cm. The contents of each tube are then sonicated using a Soniprep 150 sonicator. The tip of the probe has a diameter of approx. 3 mm and is placed just below the meniscus of the buffer. A single 10-second pulse at an amplitude of 15 μm is used to generate AXF microparticles.

Separation of AXF Sonicate Fractions

1 ml aliquots of AXF sonicate are spun at 13,000 rpm for 5 minutes in an IEC-Centra M2 bench top centrifuge. Supernatant and pellet fractions are separated and supplemented with PBS to a final volume of 1 ml. The pellet fraction is resuspended by vortexing. Preparations are used immediately.

Preparation of Immunisation Mixtures

A 1 in 4 dilution of neat AXF sonicate is chosen. This dilution has shown comparable adjuvant activity after two doses with a neat sonicate in a separate experiment that using ovalbumin as the immunogen. The highest active dilution of AXF is chosen to maximise the intramuscular injectability of the DNA formulations.

Ground particles of crosslinked AXF are used to form a 6% w/v suspension by resuspending the particles in PBS with gentle overnight stirring. The suspension is autoclaved and used immediately.

A test plasmid encoding a vaccine antigen is prepared using Qiagen Gigaprep columns in accordance with the manufacturer's instructions. Plasmid is resuspended in sterile, endotoxin-free PBS to a concentration of 1 mg/ml and frozen at −20° C. until needed.

Two doses of DNA are used in the immunisation experiment. For a dose of 10 μg, the 1 mg/ml plasmid solution is used directly. For the 1 μg dose, it is diluted 1 in 10 in PBS before use as follows:

1. PBS—80 ρl plasmid+720 μl PBS

2. Sonicate—80 μl plasmid+200 μl sonicate+520 μl PBS

3. Supernatant—80 μl plasmid+200 μl supernatant+520 μl PBS

4. Pellet—80 ml plasmid+200 μl pellet suspension+520 μl PBS

5. Ground particles—80 μl plasmid+200 μL particle suspension+520 μl PBS

6. Alum—80 μl plasmid+80 μl alum+640 μl PBS

Mixtures are vortexed briefly, with the exception of alum, which is stirred slowly for 1 hour after dropwise addition of plasmid over 30 seconds, in a modified version of the manufacturer's instructions for its use with protein immunogens (Pierce). All formulations are used as soon as possible after preparation.

9B. Immunisation and Immunoassay

50 μl of each preparation is injected into each tibialis anterior muscle of the hind legs of female C57BL/6 mice (Harlan) that are six weeks old at the first immunisation. Mice are immunised at 0, 4 and 9 weeks and bled at 3, 7 and 12 weeks.

Serum antibody titres are determined by ELISA using recombinant antigen as the coating antigen. Goat anti-mouse IgG antibody conjugated to alkaline phosphatase (Southern Biotechnology Associates) and p-nitrophenyl phosphate substrate (Sigma) in diethanolamine buffered saline are used as developing reagents. End point titres are determined by serial doubling dilution of test sera with reference to a pooled naive serum.

By following the foregoing protocol, it is found that crosslinked AXF coadministered with the plasmid DNA provides an adjuvant effect in stimulating the generation of serum antibodies to the vaccine antigen encoded by the plasmid DNA.

The foregoing experimental details are provided by way of exemplification only and are not intended to limit the scope of the invention in any way. It will readily be apparent that numerous modifications and alterations could be made to the compositions and methods set forth in the examples without departing from the principles underlying the invention and all such modifications and alterations are intended to be embraced by the claims appended hereto.

1. The use of an oxidatively cross-linked hemicellulose for the manufacture of a composition for use as a vaccine adjuvant.

2. The use of an oxidatively cross-lined hemicellulose for the manufacture of a composition for enhancing the immune response of an antigen co-administered therewith.

3. The use according to either one of claim 1 or claim 2, wherein the oxidatively cross-linked hemicellulose is an arabinoxylan, a heteroxylan or a pectin.

4. The use according to claim 3, wherein the arabinoxylan is arabinoxylan ferulate (AXF).

5. The use according to any preceding claim, wherein the oxidatively cross-linked hemicellulose is in the form of microspheres having a mean particle size of less than 50 μm (preferably less than 30 μm, more preferably less than 20 μm, for example less than 10 μm, e.g. less than 5 μm).

6. A composition comprising an oxidatively cross-linked hemicellulose as defined in any one of the preceding claims, the oxidatively cross-linked hemicellulose being in the form of microparticles having a mean particle size of less than 50 μm (preferably less than 30 μm, more preferably less than 20 μm, for example less than 10 μm, e.g. less than 5 μm).

7. A pharmaceutical composition comprising an oxidatively cross-linked hemicellulose as defined in any one of the preceding claims, wherein the oxidatively cross-linked hemicellulose is in the form of microparticles having a mean particle size of less than 50 μm (preferably less than 30 μm, more preferably less than 20 μm, for example less than 10 μm, e.g. less than 5 μm), the microparticles containing or having associated therewith a pharmaceutically useful substance.

8. A composition according to either one of claim 6 or claim 7 wherein the microparticles have a mean particle size in the range 0.5 μm to 2 μm, for example approximately 1 μm.

9. A pharmaceutical composition according to claim 7 or claim 8 (when dependent on claim 7) which is a vaccine composition, the pharmaceutically active substance being a vaccine antigen or DNA encoding a vaccine antigen.

10. A pharmaceutical composition according to claim 9 containing DNA encoding a vaccine antigen wherein the DNA is not encapsulated within the oxidatively cross-linked hemicellulose.

11. A method of preparing oxidatively cross-linked hemicellulose microparticles suitable for use in a composition as defined in any one of claims 6 to 8, which method comprises subjecting an uncrosslinked solution of a hemicellulose to oxidative gellation and comminuting the gel to give microparticles having a mean particle size of less than 50 μm.

12. A method according to claim 11, wherein the hemicellulose is AXF.

13. A method according to either one of claim 11 or claim 12, wherein the gel is dried prior to comminuting.

14. A method according to claim 13, wherein the gel is dried by freeze drying.

15. A method according to either one of claim 13 or claim 14, wherein the dried gel is comminuted to form microparticles by ball milling.

16. A method according to either one of claim 11 or claim 12, wherein the gel is comminuted whilst in a hydrated state.

17. A method according to claim 16, wherein the gel is comminuted by vortexing or sonicating.

18. A method according to any one of claims 11 to 17, wherein a pharmaceutically useful substance is mixed with the hemicellose prior to gelling and comminuting.

19. A method according to claim 18, wherein the pharmaceutically useful substance is encapsulated by the hemicellulose during the gellation.

20. A method according to any one of claims 11 to 17 wherein the gel is dried, either prior to or after comminuting, and a solution of the pharmaceutically useful substance is brought into contact with the dried gel.

21. A method of enhancing the immune response of a host to an antigen, which method comprises administering to the host, in addition to the antigen, an oxidatively cross-linked hemicellulose adjuvant as defined in any one of the preceding claims.

22. A method of stimulating an immune response to a vaccine antigen in a host (e.g. a mammalian host), which method comprises administering to the host an effective amount (e.g. an effective immune stimulating amount) of the vaccine antigen and an effective adjuvant amount of an oxidatively cross-linked hemicellulose adjuvant as defined in any one of the preceding claims.

23. A method according to claim 22, wherein the vaccine antigen and oxidatively cross-linked hemicellulose adjuvant are administered simultaneously, for example in the same composition.

24. A method of enhancing the immune response of a host to an antigen, which method comprises administering to the host a DNA construct capable of expressing the antigen in the host and a oxidatively cross-linked hemicellulose adjuvant as defined in any one of the preceding claims.

25. A method of stimulating an immune response to a vaccine antigen in a host (e.g. a mammalian host), which method comprises administering to the host an effective amount (e.g. an effective immune stimulating amount) of a DNA construct capable of expressing the antigen in the host and an effective adjuvant amount of an oxidatively cross-linked hemicellulose adjuvant as defined in any one of the preceding claims.

26. A method according to claim 24 or claim 25, wherein the DNA construct is not encapsulated within the oxidatively cross-linked hemicellulose adjuvant.

27. A method according to claim 26, wherein the DNA construct and oxidatively cross-linked hemicellulose adjuvant are coadministered in the same formulation.

28. The use of an oxidatively cross-linked hemicellulose as a vaccine adjuvant.

29. The use of an oxidatively cross-linked hemicellulose to enhance the immune response of an antigen co-administered therewith.

30 The use according to either one of claim 28 or claim 29, wherein the oxidatively cross-linked hemicellulose is an arbinoxylan, a heteroxylan or a pectin.

31. The use according to claim 30, wherein the arabinoxylan is arabinoxylan ferulate (AXF).
